(19) United States
(12) Patent Application Publication
(10) Pub. No.: US 2025/0027166 A1
(43) Pub. Date: Jan. 23, 2025

(54) SYSTEMS AND METHODS FOR THE ANALYSIS OF BIOLOGICAL SAMPLES

(71) Applicant: Gregor Diagnostics, Madison, WI (US)

(72) Inventors: Sisi Li, Madison, WI (US); Joel Wipperfurth, Madison, WI (US); Emily Breunig, Madison, WI (US); Tobias Zutz, Madison, WI (US); Tanya Quint, Madison, WI (US); Mia Shanholtzer, Madison, WI (US); D. Paul Barnes, Madison, WI (US)

(21) Appl. No.: 18/714,998

(22) PCT Filed: Nov. 30, 2022
(86) PCT No.: PCT/US2022/080669

§ 371 Date: May 30, 2024

Related U.S. Application Data
(60) Provisional application No. 63/284,389, filed on Nov. 30, 2021.

(51) Int. Cl. C12Q 1/6886; G01N 1/30; G01N 33/68

(52) U.S. Cl. CPC C12Q 1/6886; G01N 1/30; G01N 33/689; G01N 33/6893; C12Q 2600/118; C12Q 2600/154; G01N 2333/705; G01N 2333/71; G01N 2333/99; G01N 2800/50

(57) ABSTRACT

Provided herein are systems, methods, and kits for disease diagnosis or detection, including the early detection of prostate cancer in patients, diagnosing, detecting, or assigning a risk level for a disease or disorder to a subject, for disease diagnosis, monitoring, and treatment, and/or processing or preparing a sample for analysis and more particularly to methods for preparing semen samples for disease diagnosis or detection, including the detection of prostate cancer, to increase the likelihood that the sample will contain the diagnostically relevant information if the patient has a disease or is at high risk for a disease.

### CROSS-REFERENCE TO RELATED APPLICATIONS

This application claims the benefit of U.S. Provisional Application Nos. 63/284,389, filed Nov. 30, 2021, the content of which is herein incorporated by reference in its entirety.

### FIELD

Provided herein are systems and methods for disease risk assessment, detection, and diagnosis, including risk assessment and detection of prostate cancer, methods for preparing samples, including semen samples, for disease detection, diagnosis, monitoring, and treatment, and methods for semen sample analysis.

### BACKGROUND

The standard of care for prostate cancer screening is the blood Prostate Specific Antigen or PSA which measures the concentration in blood of a protein (PSA) produced by both normal and cancerous cells in the prostate. However, PSA cannot reliably tell the difference between slow-growing cancers that do not pose a risk and less common, potentially deadly cancers. This unreliability, and other factors, have led to an annual 6.5% decline in prostate cancer screening rates, while new cases and deaths continue to rise. According to the United States Preventive Services Task Force (USPSTF), 55% of biopsies taken from those with elevated PSAs were negative for prostate cancer, reaffirming the need for better, more thorough screening. These unnecessary biopsies can cause unwanted side effects including bleeding, infection, and urinary retention, leading to an additional cost and burden on the healthcare system. Furthermore, even some biopsies are unable to accurately detect prostate cancer if the sampling method misses tumor tissue.

Men diagnosed with localized or non-aggressive prostate cancers often undergo treatments with significant side effects that will not improve overall health outcomes. One disease management option for patients thought to have a low risk of complications in the absence of treatments or interventions is active surveillance. Active surveillance monitors the progression of the cancer, with the intent that if or when the disease progresses standard therapies (e.g., surgical resection, chemotherapy, or radiotherapy) can be employed. However, active surveillance is predicated on the ability to have an accurate screening procedure to allow for reliable monitoring of the progression of the cancer.

Due to concerns about overdiagnosis and overtreatment, the USPSTF discouraged PSA screening in healthy men of all ages, a “D” recommendation. The USPSTF has since upgraded the recommendation to a “C” in May 2018, advising men between the ages of 55 and 69, at average risk of developing prostate cancer, to discuss the benefits and harms of screening with their physician. However, there remains a clear need for a better screening test for prostate cancer and the ability to further characterization of the likelihood of malignant progression of such cancers.

### SUMMARY

Provided herein are systems, methods, and kits for measuring biomarkers in a sample, diagnosing, detecting, or assigning a risk level for a disease or disorder to a subject, using such information to inform care decisions, and/or processing or preparing a sample for analysis.

In some embodiments, the methods comprise collecting or receiving a sample from a subject and detecting one or more biomarkers in the sample. In some embodiments, the methods further comprise diagnosing or assigning a risk level for a disease or disorder in the subject.

In some embodiments, the sample is viscous or hyper viscous. In some embodiments, the sample is non-homogeneous. In some embodiments, the sample comprises semen, synovial fluid, mucus, pus, secretions, or combinations thereof. In some embodiments, the sample is a semen sample.

In some embodiments, the methods comprise collecting or receiving a semen sample from a subject, detecting one or more biomarkers in the semen sample, and diagnosing, detecting, or assigning a risk level for a disease or disorder in the subject.

In some embodiments, the methods further comprise drying the semen sample. In some embodiments, the sample is dried on a capture material. In some embodiments, the capture material comprises filter paper, cotton linter, or a combination thereof. In some embodiments, the methods comprise adding a buffer or buffer system to the semen sample. In some embodiments, the sample is stabilized and/or preserved by the buffer or buffer system.

In some embodiments, the drying is carried out by a device comprising a capture material. In some embodiments, the device comprises one or more sample regions each comprising capture material. In some embodiments, the capture material in each of the one or more sample regions is the same or different. In some embodiments, the drying and collecting is carried out using a single device. In some embodiments, the device further comprises a desiccant in vaporous communication with the semen sample and/or the capture material.

In some embodiments, the methods further comprise rehydrating dried semen sample. In some embodiments, the rehydrating comprises incubating the capture material with buffer or solvent at about 4° C. to about 90° C. and separating capture material from rehydrated sample. In some embodiments, the incubating is carried out at about 20° C. to about 70° C.

In some embodiments, the one or more biomarkers comprise at least one nucleic acid biomarker. In some embodiments, the at least one nucleic acid biomarker is from cell free DNA. In some embodiments, the at least one nucleic acid biomarker is selected from the group consisting of: EVX1, the gene for homeobox even-skipped homolog protein 1, FGF1, the gene for fibroblast growth factor 1, CAV1, the gene for caveolin-1, or a combination thereof.

In some embodiments, the at least one nucleic acid biomarker comprises an epigenetic marker. In some embodiments, detecting the at least one nucleic acid marker comprises determination of differential methylation of the at least one nucleic acid biomarker. In some embodiments, the differential methylation is determined by a bisulfite specific nucleic acid amplification method

In some embodiments, detecting the at least one nucleic acid biomarker comprises determining differential methylation of EVX1, CAV1, FGF1, or a combination thereof. In some embodiments, detecting the at least one nucleic acid biomarker comprises determining differential methylation of each of EVX1, CAV1, and FGF1.

In some embodiments, the at least one nucleic acid biomarker further comprises additional prostate cancer nucleic acid biomarkers. In some embodiments, the additional prostate cancer nucleic acid biomarkers comprise MCF2 (Michigan Cancer Foundation) cell line derived transforming sequence like (MCF2L), Wnt Family Member 2 (WNT2), natural cytotoxicity triggering receptor 2 (NCR2), exotosin 1 (EXT1), sperm associated antigen 4 (SPAG4), prostate cancer antigen 3 (PCA3), TMPRSS2:ETS gene fusions, glutathione S-transferase (GSTP1) methylation, CBR3 Antisense RNA 1 (CBR3-AS1), Metastasis Associated Lung Adenocarcinoma Transcript 1 (MALAT1), PCGEM1 Prostate-Specific Transcript (PCGEM1), Prostate Cancer Associated Non-Coding RNA 1 (PRNCR1), SWI/SNF Complex Antagonist Associated With Prostate Cancer 1 (SCHLAP1), and combinations thereof

In some embodiments, the at least one nucleic acid biomarker further comprises other cancer nucleic acid biomarkers or oncogenes. In some embodiments, the cancer nucleic acid biomarkers or oncogenes comprise Trafficking From ER To Golgi Regulator (TFG), Nascent Polypeptide Associated Complex Subunit Alpha (NACA), Bromodomain Containing 3 (BRD3), ETS Transcription Factor ELK4 (ELK4), Nucleophosmin 1 (NPM1), Ribosomal Protein L22 (RPL22), epithelial cellular adhesion molecule (EPCAM), Histone deacetylase 6 (HDAC6), Breast Cancer Gene 1/2 (BRCA1/2), tumor protein p53 (TP53 or p53), retinoblastoma gene (RB1), Distal-Less Homeobox 1 (DLX1), Homeobox C6 (HOXC6) or combinations thereof.

In some embodiments, the at least one nucleic acid biomarker further comprises at least one reference biomarker. In some embodiments, the reference biomarker is glyceraldehyde 3-phosphate dehydrogenase (GAPDH).

In some embodiments, the one or more biomarkers comprise at least one protein biomarker. In some embodiments, the at least one protein biomarker is selected from the group consisting of: alpha-methylacyl-CoA racemase (AMACR), Six Transmembrane Epithelial Antigen Of The Prostate 1 (STEAP1), Growth Hormone Secretagogue Receptor (GHSR), prostate-specific membrane antigen (PSMA), and combinations thereof. In some embodiments, detecting the at least one protein biomarker comprises determining the expression level of the at least one protein biomarker in an immunoassay.

In some embodiments, the at least one protein biomarker further comprises prostate-specific antigen (PSA), endoglin, prostate-specific membrane antigen (PSMA), caveolin-1, interleukin-6, cluster of differentiation 147 (CD147), Transforming growth factor beta 1 (TGF-β1), human kallikrein-2, or combination thereof.

In some embodiments, the at least one protein biomarker further comprises at least one reference biomarker. In some embodiments, the reference protein biomarker comprises matriptase.

In some embodiments, the protein biomarker is associated with a cellular component or fragment thereof (e.g., an organelle, a vesicle, or a fragment thereof). In some embodiments, the at least one protein biomarker is a bioparticle surface protein. Thus, in some embodiments, the one or more biomarkers comprise at least one bioparticle surface biomarker.

In some embodiments, the methods further comprise quantifying bioparticles in the sample. In some embodiments, detecting the at least one protein biomarker comprises quantifying bioparticles with STEAP1, GHSR, PSMA, or a combination thereof. In some embodiments, detecting the at least one protein biomarker comprises quantifying bioparticles with STEAP1, GHSR, and PSMA.

Thus, in some embodiments, the methods comprise collecting or receiving a sample from a subject, detecting one or more biomarkers in the sample, wherein the one or more biomarkers comprise: at least one protein biomarker comprises alpha-methylacyl-CoA racemase (AMACR), at least one bioparticle surface marker selected from the group consisting of STEAP1, GHSR, PSMA, and combinations thereof, at least one nucleic acid biomarker selected from the group consisting of EVX1, CAV1, FGF1, or a combination thereof, or any combination thereof.

In some embodiments, the one or more biomarkers comprises at least one protein biomarker and at least one nucleic acid biomarker. In some embodiments, the one or more biomarkers comprises at least one protein biomarker and at least one bioparticle surface marker. In some embodiments, the one or more biomarkers comprises at least one nucleic acid biomarker and at least one bioparticle surface marker. In some embodiments, the one or more biomarkers comprise at least one protein biomarker, at least one nucleic acid biomarker, and at least one bioparticle surface marker.

In some embodiments, detecting one or more biomarkers in the sample comprises detecting the expression level of AMACR, quantifying bioparticles with STEAP1, GHSR, and PSMA, determining differential methylation of each of EVX1, CAV1, and FGF1, and any combination thereof. In some embodiments, detecting one or more biomarkers in the semen sample comprises detecting the expression level of AMACR, quantifying bioparticles with STEAP1, GHSR, and PSMA, and determining differential methylation of each of EVX1, CAV1, and FGF1.

In some embodiments, the methods further comprise detecting one or more control biomarkers.

In some embodiments, diagnosing or assigning a risk level for a disease or disorder comprises providing a risk score based on evaluation of the one or more biomarkers. In some embodiments, the disease or disorder comprises cancer (e.g., prostate cancer). In some embodiments, the methods further comprise treating the subject.

In some embodiments, the methods further comprise shipping the sample (e.g., the semen sample or biological sample). In some embodiments, shipping the sample is carried out using the same device as the drying and collecting. In some embodiments, the sample at least partially dries during shipping.

Also provided herein are methods for processing or preparing a sample for analysis. In some embodiments, the methods comprise collecting or receiving a sample from a subject, drying the sample, and rehydrating dried sample.

In some embodiments, the sample is viscous or hyper viscous. In some embodiments, the sample is non-homogeneous. In some embodiments, the sample comprises semen, synovial fluid, mucus, pus, secretions, urine, blood, or combinations thereof. In some embodiments, the sample is a semen sample.

In some embodiments, the sample is dried on a capture material. In some embodiments, the capture material comprises filter paper, cotton linter, an ionic or adsorption membrane, a microparticle assembly, or a combination thereof.

In some embodiments, the drying is carried out by a device comprising a capture material. In some embodiments, the device comprises one or more sample regions each comprising capture material. In some embodiments, the capture material in each of the one or more sample regions is the same or different. In some embodiments, the drying and collecting is carried out using by a single device. In some embodiments, the device further comprises a desiccant in vaporous communication with the semen sample and/or the capture material.

In some embodiments, the rehydrating comprises: incubating the capture material with buffer or solvent at about 4° C. to about 90° C. and separating capture material from rehydrated sample. In some embodiments, the incubating is carried out at about 20° C. to about 70° C.

In some embodiments, the methods further comprise shipping the sample (e.g., the semen sample or biological sample). In some embodiments, shipping the sample is carried out using the same device as the drying and collecting. In some embodiments, the sample at least partially dries during shipping.

In some embodiments, the methods further comprise analyzing the rehydrated sample. In some embodiment, the analyzing comprises detecting one or more biomarkers in the rehydrated biological sample.

In some embodiments, the one or more biomarkers comprise at least one nucleic acid biomarker. In some embodiments, the at least one nucleic acid biomarker is from cell free (cfDNA) or circulating tumor (ctDNA) or circulating tumor cells (CTCs). In some embodiments, the at least one nucleic acid biomarker is selected from the group consisting of: EVX1, CAV1, FGF1, or a combination thereof.

In some embodiments, the at least one nucleic acid biomarker comprises an epigenetic marker. In some embodiments, detecting the at least one nucleic acid marker comprises determination of differential methylation of the at least one nucleic acid biomarker. In some embodiments, the differential methylation is determined by a bisulfite specific nucleic acid amplification method.

In some embodiments, detecting the at least one nucleic acid marker comprises determining differential methylation of EVX1, CAV1, FGF1, or a combination thereof. In some embodiments, detecting the at least one nucleic acid marker comprises determining differential methylation of each of EVX1, CAV1, and FGF1.

In some embodiments, the one or more biomarkers comprise at least one protein biomarker. In some embodiments, the at least one protein biomarker is selected from the group consisting of: alpha-methylacyl-CoA racemase (AMACR), STEAP1, GHSR, PSMA, and combinations thereof. In some embodiments, detecting the at least one protein biomarker comprises determining the expression level of the at least one protein biomarker in an immunoassay.

In some embodiments, the at least one protein biomarker is a bioparticle surface protein. Thus, in some embodiments, the one or more biomarkers comprise at least one bioparticle surface biomarker.

In some embodiments, the methods further comprise quantifying bioparticles in the sample. In some embodiments, detecting the at least one protein biomarker comprises quantifying bioparticles with STEAP1, GHSR, PSMA, or a combination thereof. In some embodiments, detecting the at least one protein biomarker comprises quantifying bioparticles with STEAP1, GHSR, and PSMA.

Thus, in some embodiments, the methods comprise collecting or receiving a sample from a subject, detecting one or more biomarkers in the sample, wherein the one or more biomarkers comprise: at least one protein biomarker comprising alpha-methylacyl-CoA racemase (AMACR), at least one bioparticle surface marker selected from the group consisting of STEAP1, GHSR, PSMA, and combinations thereof, at least one nucleic acid biomarker selected from the group consisting of EVX1, CAV1, FGF1, or a combination thereof, or any combination thereof.

In some embodiments, the one or more biomarkers comprise at least one protein biomarker, at least one nucleic acid biomarker, and at least one bioparticles surface marker. In some embodiments, detecting one or more biomarkers in the semen sample comprises detecting the expression level of AMACR, quantifying bioparticles with STEAP1, GHSR, and PSMA, determining differential methylation of each of EVX1, CAV1, and FGF1, and any combination thereof. In some embodiments, detecting one or more biomarkers in the semen sample comprises detecting the expression level of AMACR, quantifying bioparticles with STEAP1, GHSR, and PSMA, and determining differential methylation of each of EVX1, CAV1, and FGF1.

In some embodiments, the methods further comprise detecting one or more control biomarkers.

In some embodiments, the methods further comprise diagnosing or assigning a risk level for a disease or disorder in the subject. In some embodiments, diagnosing or assigning a risk level for a disease or disorder comprises providing a risk score based on evaluation of the one or more biomarkers.

### BRIEF DESCRIPTION OF THE DRAWINGS

FIG. 1 is a schematic of exemplary assays and analysis processes.

FIG. 2 is graphs of initial analysis of sensitivity of an exemplary biomarker panel compared to PSA-only.

FIG. 3 shows the results from an exemplary biomarker panel prediction in comparison to biopsy confirmation and the relative sensitivity and specificity for cancer detection and differentiation.

FIG. 4 is a contingency table for an exemplary panel prediction in comparison to biopsy confirmation showing results for negative, low-risk, intermediate risk, and high-risk subjects.

### DETAILED DESCRIPTION

The present disclosure provides methods related to disease diagnosis and sample preparation and analysis.

Section headings as used in this section and the entire disclosure herein are merely for organizational purposes and are not intended to be limiting.

### 1) Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

A “biomarker” includes a biological compound, such as a protein and a fragment thereof, a peptide, a polypeptide, a proteoglycan, a glycoprotein, a lipoprotein, a carbohydrate, a lipid, a nucleic acid, an organic on inorganic chemical, a natural polymer, a cell fragment, an exosome, and a small molecule, that is present in a biological sample and that may be isolated from, or measured in, the biological sample. Furthermore, a biomarker may be the entire intact molecule, or a portion thereof that may be partially functional or recognized, for example, by an antibody or other specific binding protein. A biomarker may be associated with a given state of a subject, such as a particular stage of disease. In some embodiments, the biomarker is a cancer biomarker (e.g., circulating tumor DNA, protein biomarkers (e.g., prostate specific antigen, alpha-fetoprotein, carcinoembryonic antigen). A measurable aspect of a biomarker may include, for example, the presence, absence, or concentration of the biomarker in the biological sample from the subject and/or relative changes of any of the measurable aspects compared to a standard (e.g., internal or from a healthy subject). The measurable aspect may also be a ratio of two or more measurable aspects of two or more biomarkers. Biomarker, as used herein, also encompasses a biomarker profile comprising measurable aspects of two or more individual biomarkers. The two or more individual biomarkers may be from the same or different classes of biomarkers such as, for example, a nucleic acid and a carbohydrate, or may measure the same or different measurable aspect such as, for example, absence of one biomarker and concentration of another. A biomarker profile may comprise any number of individual biomarkers or features thereof. In another embodiment, the biomarker profile comprises at least one measurable aspect of at least one internal standard. Methods of identifying and quantifying biomarkers are well known in the art and include histological and molecular methods such as enzyme-linked immunosorbent assays (ELISA) and other immunoassays, gel electrophoresis protein and DNA arrays, mass spectrometry, colorimetric assays, electrochemical assays, and fluorescence methods.

The terms “buffer” or “buffer system” are used herein to refer to a compound or compounds that provide a solution that exhibits buffering capacity, that is, the capacity to neutralize, within limits, either acids or bases (alkali) with relatively little or no change in the original pH. Buffer characteristics are defined in detail in, for example, Remington's Pharmaceutical Sciences, 18th Ed. Gennaro Ed., Mack Publishing Co., Easton Pa. (1990) pp. 241-243 Exemplary buffer systems include acetate, succinate, citrate, prolamine, histidine, borate, carbonate and phosphate buffers and buffer systems. In some embodiments, the buffer or buffer system, when added to the samples (e.g., semen samples), stabilizes and/or preservers the sample.

As used herein, “methylation” refers to any and all processes by which methyl group(s) are added to a nucleic acid. For example, methylation may include, but is not limited to, the addition of methyl groups at positions C5 or N4 of cytosine or at the N6 position of adenine. Accordingly, as used herein a “methylated nucleotide base” or “methylated nucleotide” refers to the presence of a methyl moiety on a nucleotide base, where the methyl moiety is not present in a recognized canonical nucleotide base. For example, cytosine does not contain a methyl moiety on its pyrimidine ring, but 5-methylcytosine contains a methyl moiety at position 5 of its pyrimidine ring. As used herein, a “methylated nucleic acid” refers to a nucleic acid molecule that contains one or more methylated nucleotides. A nucleic acid molecule containing a methylated cytosine is considered methylated (e.g., the methylation state of the nucleic acid molecule is methylated). A nucleic acid molecule that does not contain any methylated nucleotides is considered unmethylated. As used herein, a “methylation profile,” “methylation status,” “methylation state,” and “methylation signature” of a nucleic acid molecule all refer to the presence of absence of one or more methylated nucleotide bases in the nucleic acid molecule. A change in the methylation profile may be due to presence of methylation at a site or sites which is normally non-methylated, or alternatively, may be due to the absence of methylation at a site or sites which is normally methylated. A change in the methylation profile at a single site may or may not be indicative of a disease state.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

As used herein, the term “sample” is used in its broadest sense. In one sense, it is meant to include a specimen obtained from any source, including biological and environmental samples. Biological samples may be obtained from animals (including humans) and encompass fluids, solids, tissues, and gases. Such examples are not however to be construed as limiting the sample types. Preferably, a sample is a fluid sample such as a liquid sample. Examples of liquid samples that may be assayed include bodily fluids (e.g., blood, serum, plasma, saliva, urine, ocular fluid, semen, sputum, sweat, tears, and spinal fluid), water samples (e.g., samples of water from oceans, seas, lakes, rivers, and the like), samples from home, municipal, or industrial water sources, runoff water, or sewage samples; and food samples (e.g., milk, beer, juice, or wine). Viscous liquid, semisolid, or solid specimens may be used to create liquid solutions, eluates, suspensions, or extracts that can be samples. For example, throat or genital swabs may be suspended in a liquid solution to make a sample. Samples can include a combination of liquids, solids, gasses, or any combination thereof (e.g., a suspension of lysed or unlysed cells in a buffer or solution). Samples can comprise biological materials, such as cells, microbes, organelles, and biochemical complexes. Liquid samples can be made from solid, semisolid, or highly viscous materials, such as soils, fecal matter, tissues, organs, biological fluids, or other samples that are not fluid in nature. For example, solid or semisolid samples can be mixed with an appropriate solution, such as a buffer, a diluent, and/or extraction buffer The sample can be macerated, frozen and thawed, or otherwise extracted to form a fluid sample. Residual particulates may be removed or reduced using conventional methods, such as filtration or centrifugation.

A “subject” may be human or non-human and may include either adults or juveniles (e.g., children). Moreover, subject may mean any living organism, preferably a mammal (e.g., human or non-human). Examples of mammals include, but are not limited to, any member of the Mammalian class: humans, non-human primates such as chimpanzees, and other apes and monkey species, farm animals such as cattle, horses, sheep, goats, swine; domestic animals such as rabbits, dogs, and cats; laboratory animals including rodents, such as rats, mice and guinea pigs, and the like. In one embodiment of the methods and compositions provided herein, the mammal is a human. “Subject” and “patient” may be used interchangeably herein.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting. Illustrative embodiments of the invention are shown in the figures. It should be understood that the invention is not limited to these particular examples.

### 2) Methods

The technology relates to systems, methods, and kits for measuring biomarkers (e.g., one or more different types of biomarker) in a sample, diagnosing, detecting, or assigning a risk level for a disease or disorder to a subject, for disease diagnosis, monitoring, and treatment, and/or processing or preparing a sample for analysis.

In some embodiments, the methods comprise at least one or more or all of collecting or receiving a sample from a subject, drying the sample, rehydrating the sample, shipping the sample, detecting one or more biomarkers in the samples, diagnosing, detecting, or assigning a risk level for a disease or disorder in a subject, and treating a subject.

a. Samples

A number of different samples are suitable with the present technology. In some embodiments, the sample is a viscous or hyper viscous sample. Exemplary viscous samples include biological fluids and samples comprising semen, synovial fluid, mucus (e.g., cervical mucus, respiratory mucus), pus, secretions (e.g., prostate secretions) and the like. Some biological fluids, such as blood, vary in viscosity and increased viscosity may be an indication of a disease, disorder, or abnormality. The methods described herein would be equally applicable viscous or non-viscous samples, thereby allowing use of the same methods for an individual sample type regardless of viscosity.

In some embodiments, the sample is non-homogeneous. Non-homogeneous describes samples that are not uniform in content (e.g., homogeneous) throughout the sample, as well as samples that contain variable components, for example, components may or may not be consistently present in the sample. For example, sample contamination with other cell types or components from surrounding tissues and fluids may only be present in the sample occasionally, resulting in difficulties processing under standard conditions. The present disclosure provides methods applicable with these more complex and inconsistent samples.

Thus, in some embodiments, the methods described herein assist in handling and process techniques for high viscosity, potentially complex, and/or non-homogeneous samples.

In some embodiments of the technology, the sample is a biological sample. Biological samples include but are not limited to a sample from a subject such as an animal (e.g., a mammal (e.g., a primate (e.g., human))). A sample from a subject can be of any appropriate type, such as a sample of fluid, tissue, organ, or a combination thereof. Exemplary biological samples, include but are not limited to fluid samples (e.g., urine, blood, serum, saliva, semen, secretions (e.g., vaginal secretions), central nervous system fluids, lavages, and the like). In some embodiments, the sample is semen. The biological sample can also be a sample of other biological material, such as food, including food materials derived from plants or animals or combinations thereof.

Any of the biological samples described herein may be obtained from the subject using any known technique.

The methods are suitable for any volume of sample. In some embodiments, the sample is 0.01-100 mL. In some embodiments, the sample is low volume (e.g., less than 1 mL, less than 0.5 mL, less than 0.1 mL, less than 0.01 mL). In some embodiments, the sample volume is greater than approximately 0.01 mL, 0.1 mL, 0.25 mL, 0.5 mL, 1 mL, 2, mL, 3 mL, 4 mL, 5 mL, 6 mL, 7 mL, 8 mL, 9 mL, 10 mL, or more. In some embodiments, the sample volume is 0.25-20 mL, 0.25-15 mL, 0.25-10mL, 0.25-5 mL, 0.5-20 mL, 0.5-15 mL, 0.5-10mL, 0.5-5 mL, 0.1-20 mL, 0.1-15 mL, 0. 1-10mL, 0.1-5 mL, 5-20 mL, 5-15 mL, or 5-10 mL.

In some embodiments, the methods comprise adding a buffer or buffer system to the sample. In some embodiments, the sample is stabilized and/or preserved by the buffer or buffer system.

In some embodiments, the sample is a dried or desiccated sample. Accordingly, in some embodiments, the methods further comprise drying the sample. The sample may be dried at any temperature or drying condition (low pressure or elevated temperature), with or without the use of drying agents (e.g., desiccant, diatomaceous earth, clays, etc.). In some embodiments, drying preserves the sample for analysis at a wide range of temperatures without significant degradation.

In some embodiments, the sample is dried on a capture material. The capture material may be any matrix or structure designed to capture analytes or subgroups of analytes either specifically or non-specifically. The capture material may comprise a wide variety of adsorbent or absorbent materials. For example, the capture material may comprise modified or unmodified membranes (e.g., PVDF/nitrocellulose), chromatography resins, filter materials (e.g., filter papers or nylon filter material), cloth, cotton linter, synthetic knits (e.g., polyester hydro knit), clays, polypropylene absorbents, microparticles, or combinations thereof. In some embodiments, the capture material comprises filter paper, cotton linter, or a combination thereof.

In some embodiments, the drying is carried out by a device comprising the capture material(s) as a sub-component thereof. In some embodiments, the device comprises one or more sample regions each comprising a capture material. In some embodiments, the capture material is the same in each of the sample regions. Alternatively, the capture material may be different in one or more of the sample regions.

In some embodiments, the capture material is configured for removal from sample regions, and recovery and analysis of the captured, dried sample. The quantity of capture material can be optimized based on the amount of sample in each of the one or more sample regions and the relative loading capacity of the capture material. The capture material can be sized accordingly to fit within the sample regions.

In some embodiments, the device further comprises a desiccant in vaporous communication with the capture material and/or sample to facilitate drying of the sample on the capture material. In some embodiments, the desiccant is held in place in the cap with an air permeable barrier, structure, or chamber.

Exemplary desiccants include natural or synthetic desiccants including anhydrous calcium sulfate (e.g., gypsum), aluminas, including activated aluminas (e.g., aluminum oxide or Al₂O₃), glass, silicas (e.g., SiO₂, silica gels, Ascarite II® absorbents (e.g., carbon dioxide adsorbents including sodium hydroxide-coated silica), or diatomaceous silicas (e.g., Celite®, Celatom®, CAFA (Celite® Analytical Filter Aid))), a hygroscopic polymer and/or salt (e.g., CaCl₂, CaO, ZnCl₂, KOH, NaOH, CaH₂, CaSO₄, and Na₂SO₄), molecular sieves or crystalline metal aluminosilicates, activated carbon, montmorillonites or montmorillonite clays (e.g., (Al₂O₃·4SiO₂·xH₂O)), and drying agents (e.g., barium oxide, boron oxide, calcium salts (e.g., calcium chloride, calcium oxide, calcium hydride), copper (II) sulfate, lithium aluminum hydride, lithium chloride, magnesium oxide, magnesium perchlorate, magnesium sulfate, phosphorus pentoxide, potassium hydroxide, sodium, sodium hydroxide, or sodium-potassium alloy). In preferred embodiments, the desiccant is montmorillonite clay, lithium chloride, activated alumina, alkali aluminosilicate, DQ11 Briquettes, silica gel, molecular sieve, calcium sulfate, or calcium oxide. In some embodiments, the desiccant comprises a silica gel, molecular sieve, aluminosilicates, or a combination thereof.

The quantity or surface area of desiccant can be optimized to adjust the drying time of the sample. In some embodiments, the amount and surface area of desiccant is sufficient to dry the sample in less than 30 minutes, 1 hour, 2 hours, 3 hours, 4 hours, 6 hours, 12 hours, 24 hours, 36 hours, or 48 hours.

In some embodiments, the drying and collecting is carried out using by a single device. The device may comprise a collection chamber to receive and hold the sample. The device may further comprise a mechanism to apply the sample to the capture materials. For example, the device may facilitate dipping capture materials into the sample in the collection chamber, or the device may passively or actively transfer sample to compartments within the device comprising the capture materials, or the sample may be wicked onto the capture materials, or the capture materials may be provided on a removable section of the device that allows dosing onto multiple capture materials from a single port.

In some embodiments, the methods further comprise rehydrating the dried samples. For example, the methods may comprise collecting or receiving the sample, drying the sample, and rehydrating the sample.

In some embodiments, the rehydrating comprising incubating the capture material with a buffer or solvent at a defined temperature about 4° C. to about 90° C. for a period of time and separating the capture material from the rehydrated sample. In some embodiments, the incubating is carried out at about 20° C. to about 70° C. In select embodiments, the incubating is carried out at about 4° C., about 10° C., about 15° C., about 20° C., about 25° C., about 30° C., about 35° C., about 40° C., about 45° C., about 50° C., about 55° C., about 60° C., about 65° C., about 70° C., about 75° C., or about 80° C.

The incubation may be carried out and optimized for the time necessary to allow at least partial rehydration of the sample. The total time is dependent on type and quantity of the sample, type and quantity of the capture material, and solvent or buffer.

In some embodiments, the incubation time is 1-60 minutes (e.g., 5-50 minutes, 10-40 minutes, 20-30 minutes). In some embodiments, the incubation time is about 1 min, about 5 min, about 10 min, about 15 min, about 20 min, about 25 min, about 30 min, about 35 min, about 40 min, about 45 min, about 50 min, about 55 min, or about 60 min.

The rehydration buffer or solvent may be any of those that allow rehydration and are compatible with subsequent downstream processing (e.g., detecting and/or measuring any of the disclosed biomarkers). Exemplary buffers and solvents suitable for use with the methods disclosed herein include, but are not limited to, water, isotonic saline, phosphate buffered saline (PBS), 4-2-hydroxyethyl-1-piperazineethanesulfonic acid buffer (HEPES), 3-(N-morpholino)propanesulfonic acid buffer (MOPS), and piperazine-N,N′-bis(2-ethanesulfonic acid) buffer (PIPES), Tris buffered saline (TBS), tromethamine hydrochloride, Hanks' balanced salt solution, Earle's balanced salt solution, and Ringer's solution.

Rehydrating the samples as described herein results in more homogeneous samples compared to the same samples recovered by centrifugation, thereby allowing consistent processing of more complex, viscous samples not previously achievable.

The rehydrated sample may be separated from the capture material using methods known in the art (e.g., centrifugation, magnetic separation, immobilization etc.). The rehydrated sample may be used directly or following a pretreatment step to modify the character of the sample. Such pretreatment step may include, for example, filtration, precipitation, dilution, distillation, mixing, concentration, inactivation or removal of interfering components, the addition of reagents, lysing, and the like.

In some embodiments, the methods further comprise shipping the sample, e.g., shipping the device to a testing location. In some embodiments, the sample is collected outside of a clinical setting (e.g., at home) or in a clinical setting and shipped to the test location. In some embodiments, the shipping is carried out using the same device as the drying and collecting. In some embodiments, the device is packaged in a shipping container directly. Alternatively, in some embodiments, the device acts as a shipping container.

The samples may be shipped using any known methods to transport a sample, including any temperature condition. In some embodiments, the samples are shipped at ambient or room temperature (e.g., 15-30° C.). In some embodiments, the samples are shipped at cool or cold temperatures, below that of ambient or room temperature (e.g., 2-15° C., −20° C. or lower).

In some embodiments, the sample at least partially dries during shipping. In some embodiments, the sample is at least partially dried prior to shipping. Thus, the sample may arrive at the testing location fully dried.

In some embodiments, the methods further comprise storing the dried sample. For example, the sample may be stored for a period of several minutes, hours, days, months, or more. In some embodiments, the storing is carried out using the same device as the drying, collecting, and shipping. In some embodiments, the samples are stored at ambient or room temperature (e.g., 15-30° C.). In some embodiments, the sample are stored at cool temperatures, below that of ambient or room temperature (e.g., 2-15° C., −20° C. or lower).

Drying the sample may confer increased stability for storage. In some embodiments, the sample when dried is stable for up to 7 days at elevated temperatures above room temperature (e.g., 50-60° C.). In some embodiments, the dried sample is stable for over 1 week, over 1 month, over 6 months, over 1 year when frozen.

b. Analyzing the Sample

In some embodiments, the methods may further comprise analyzing the sample (e.g., the dried and rehydrated sample). The analysis can include any assay useful, necessary, or sufficient to characterize the sample. In some embodiments, the analysis comprises conducting one or more biological assays with the sample. In some embodiments, the biological assay detects the presence, absence, or quantity of at least one biomarker (e.g., a cancer biomarker, e.g., a prostate cancer biomarker).

In some embodiments, the analyzing comprises detecting one or more biomarkers in the sample. In some embodiments, the methods comprise collecting or receiving a sample from a subject and detecting one or more biomarkers in the sample.

Any method suitable for determining the amount of the biomarkers may be used herein. Methods of identifying and quantifying biomarkers include histological and molecular methods such as enzyme-linked immunosorbent assays (ELISA) and other immunoassays, electrophoresis methods (e.g., gel electrophoresis or capillary electrophoresis), protein and DNA arrays, DNA amplification based methods (e.g., PCR, RT-PCR, methylation-specific PCR, isothermal amplification), DNA hybridization, mass spectrometry, spectrophotometry, spectroscopy, colorimetric assays, electrochemical assays, analytical chromatography methods, and fluorescence methods.

In some embodiments, the one or more biomarkers comprise at least one protein biomarker, at least one nucleic acid biomarker, or a combination thereof. In some embodiments, the biomarkers may be indicative of a disease (e.g., disease-related biomarkers) or general patient health (e.g., genomic, proteomic, metabolomic data). In some embodiments, the at least one biomarker comprises disease-associated (e.g., cancer-associated) genes. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence, risk of, and/or progression of the disease. A disease-associated gene also refers to a gene, the mutation or genetic variation of which is directly responsible or is in linkage disequilibrium with a gene(s) that is responsible for the etiology of a disease.

The nucleic acid biomarker may be from any type of nucleic acid, including but not limited to, genomic DNA, circulating free or cell free DNA, circulating tumor DNA, RNA (e.g., mRNA, miRNA, siRNA, and the like) or any combination thereof. In some embodiments, the at least one nucleic acid biomarker is from cell free DNA. In some embodiments, the at least one nucleic acid biomarker is cellular DNA (e.g., from exfoliated cells in urine or semen).

In some embodiments, the nucleic acid biomarker comprises one or more mutations, one or more translocations, one or more deletions, one or more insertions, one or more epigenetic markers or a combination thereof. In some embodiments, the biological assay detects the presence or absence of one or more mutations, one or more translocations, one or more deletions, one or more insertions, or one or more epigenetic markers in the nucleic acid biomarker. In some embodiments, the biological assay detects the quantity (e.g., copy number or expression level) of the nucleic acid biomarker.

In some embodiments, the nucleic acid biomarker comprises EVX1, the gene for homeobox even-skipped homolog protein 1, FGF1, the gene for fibroblast growth factor 1, CAV1, the gene for caveolin-1, and combinations thereof.

In some embodiments, the nucleic acid biomarker comprises an epigenetic marker or modification, including chemical modifications (e.g., methylation). Methylation, particularly at the 5 position of cytosine, is a major epigenetic modification in the mammalian genome that is associated with biological and pathological processes, including as well-accepted biomarkers of various diseases. Accordingly, the methods further comprise determination of changes in epigenetic markers or modifications in the at least one nucleic acid biomarker.

In some embodiments, the methods further comprise determination of differential methylation or methylation profile of the at least one nucleic acid biomarker. Thus, in some embodiments, the methods further comprise determination of differential methylation or a methylation profile of EVX1, FGF1, CAV1, or a combination thereof.

In some embodiments, the methods further comprise identifying or detecting one or more methylated nucleotides in the nucleic acid biomarker. Methylation may be determined using known methods, for example, by methylation specific PCR, whole-genome, base-resolution, and quantitative sequencing methods, incorporating methods such as bisulfite sequencing and/or differential hybridization of detection probes. Determination of the differential methylation or a methylation profile may target a single region of the nucleic acid biomarker, multiple regions of the nucleic acid biomarker, or the entire biomarker.

In some embodiments, the methods further comprise determining whether the differential methylation is indicative of a disease or disorder. In some embodiments, the differential methylation comprises a differentially methylated region (DMR) or a different methylation profile in the diseased subject versus a healthy subject. In some embodiments, the method further comprises classifying the sample and/or subject based on the DMR or methylation status as compared to a reference. The differential methylation may comprise hypomethylation or hypermethylation in the DMR. As such, the differential methylation may comprise additional methylation not present in a normal, non-diseased sample or, alternatively, the differential methylation may lack methylation present in a normal, non-diseased sample.

In some embodiments, the at least one nucleic acid biomarker further comprises additional prostate cancer nucleic acid biomarkers. In some embodiments, the additional prostate cancer nucleic acid biomarkers comprise MCF2 (Michigan Cancer Foundation) cell line derived transforming sequence like (MCF2L), Wnt Family Member 2 (WNT2), natural cytotoxicity triggering receptor 2 (NCR2), exotosin 1 (EXT1), sperm associated antigen 4 (SPAG4), prostate cancer antigen 3 (PCA3), TMPRSS2:ETS gene fusions, glutathione S-transferase (GSTP1) methylation, CBR3 Antisense RNA 1 (CBR3-AS1), Metastasis Associated Lung Adenocarcinoma Transcript 1 (MALAT1), PCGEM1 Prostate-Specific Transcript (PCGEM1), Prostate Cancer Associated Non-Coding RNA 1 (PRNCR1), SWI/SNF Complex Antagonist Associated With Prostate Cancer 1 (SCHLAP1), and combinations thereof

In some embodiments, the at least one nucleic acid biomarker further comprises other cancer nucleic acid biomarkers or oncogenes. In some embodiments, the cancer nucleic acid biomarkers or oncogenes comprise Trafficking From ER To Golgi Regulator (TFG), Nascent Polypeptide Associated Complex Subunit Alpha (NACA), Bromodomain Containing 3 (BRD3), ETS Transcription Factor ELK4 (ELK4), Nucleophosmin 1 (NPM1), Ribosomal Protein L22 (RPL22), epithelial cellular adhesion molecule (EPCAM), Histone deacetylase 6 (HDAC6), Breast Cancer Gene 1/2 (BRCA1/2), tumor protein p53 (TP53 or p53), retinoblastoma gene (RB1), Distal-Less Homeobox 1 (DLX1), Homeobox C6 (HOXC6) or combinations thereof.

A “protein biomarker,” as used herein, is a biomarker associated with a protein. Examples of a protein biomarker include, but are not limited to, a protein expression level (e.g., an increased expression level or a decreased expression level), a protein activity level (e.g., an increased activity level or a decreased activity level), a protein mutation, protein localization, a post-translational modification, and a protein truncation.

In some embodiments, the at least one protein biomarker is selected from the group consisting of alpha-methylacyl-CoA racemase (AMACR), Six Transmembrane Epithelial Antigen Of The Prostate 1 (STEAP1), Growth Hormone Secretagogue Receptor (GHSR), prostate-specific membrane antigen (PSMA), and combinations thereof. In some embodiments, the at least one protein biomarker comprises each of alpha-methylacyl-CoA racemase (AMACR), Six Transmembrane Epithelial Antigen Of The Prostate 1 (STEAP1), Growth Hormone Secretagogue Receptor (GHSR), and prostate-specific membrane antigen (PSMA).

In some embodiments, the methods further comprise identifying if the protein biomarker is differentially expressed and determining whether the differential expression is indicative of a disease or disorder. The phrase “differentially expressed” refers to differences in the quantity and/or the frequency of a biomarker present in a sample taken from the subject having or at risk of a disease or disorder compared to a control subject. For example, the protein biomarkers may be present at an elevated level or at a decreased level in samples of a subject having or at risk of a disease or disorder compared to samples of control subjects. A biomarker can be differentially present in terms of quantity, frequency, or both.

Expression levels of the protein biomarkers can be determined by any suitable method known in the art. In one embodiment, expression levels of the protein biomarkers are determined by measuring polypeptide levels of the biomarkers. Assays based on the use of antibodies that specifically recognize the proteins or polypeptide fragments of the biomarkers may be used for the measurement. Such assays include, but are not limited to, immunohistochemistry (IHC), flow cytometry, cytometry by time of flight (CyTOF), western blotting, enzyme-linked immunosorbent assay (ELISA), radioimmunoassays (RIA), “sandwich” immunoassays, fluorescent immunoassays, enzyme multiplied immunoassay technique (EMIT), capillary electrophoresis immunoassays (CEIA) immunoprecipitation assays, the procedures of which are well known in the art (see, e.g., Ausubel et al, eds, 1994, Current Protocols in Molecular Biology, Vol. 1, John Wiley & Sons, Inc., New York, which is incorporated by reference herein in its entirety).

In some embodiments, the protein biomarker is associated with a cellular component or fragment thereof. For example, the protein biomarker may be within or associated with an organelle or a vesicle (e.g., extracellular vesicles, microvesicles, apoptotic bodies, intracellular vesicles).

In some embodiments, the at least one protein biomarker is a bioparticle surface protein. As used herein, the term “bioparticle” is used to refer to any subcellular particle derived from a biological system or comprised of biological materials, e.g., cell components or fragments thereof. The bioparticle can be proteinaceous, nucleic acid-containing, lipid-containing, steroidal, or a combination thereof. The bioparticles may comprise organelles or organelle fragments.

The bioparticles may take on many forms. In some embodiments, the bioparticles may be vesicular in nature having a lipid layer or bilayer, in some instances separating an internal compartment from the external environment. In some embodiments, the bioparticles may be comprised of aggregates of biomolecules (e.g., proteins, lipids, nucleic acids, or carbohydrates) or other bioparticles.

The bioparticles may be various sizes. In some embodiments, the bioparticles are less than 10 μm, less than 5 μm, less than 2 μm, or less than 1 μm. In some embodiments, the bioparticles range from about 1 nm to about 5 μm, about 1 nm to about 2 μm, about 1 nm to about 1 μm, about 10 nm to about 5 μm, about 10 nm to about 2 μm, about 10 nm to about 1 μm, about 100 nm to about 5 μm, about 100 nm to about 2 μm, or about 100 nm to about 1 μm.

As such, in some embodiments, the one or more biomarkers may include at least one bioparticle surface marker. In some embodiments, the at least one bioparticle surface marker comprises STEAP1, GHSR, PSMA, and combinations thereof.

The methods may further comprise quantifying the bioparticles in the sample. In some embodiments, the methods may further comprise quantifying the bioparticles in the sample comprising at least one or all of the target bioparticle surface markers. In some embodiments, the methods comprise quantifying the bioparticles in the sample comprising all of the target bioparticle surface markers. In some embodiments, the methods further comprise determining whether an increase in any or all of the at least one bioparticle surface marker is indicative of a disease or disorder when compared to a control, non-diseased, sample.

In some embodiments, the methods further comprise determining whether a change in the quantity of bioparticles may be an indication of a subject with a disease or disorder. For example, bioparticles may be present at an elevated level or at a decreased level in samples of a subject having or at risk of a disease or disorder compared to samples of control subjects.

Bioparticle characterization can be done using methods including but not limited to flow methods such as flow cytometry and microfluidic sorting. Detection, characterization, and, accordingly, sorting may be conducted based on any of bioparticle size, the fluorescence spectrum of an attached reporter molecule (e.g., an antibody which specifically detects a bioparticle surface marker comprising a detectable moiety (e.g., a fluorophore)), the fluorescence intensity of the reporter molecule, and the number of bioparticle in each classification bin.

In some embodiments, the at least one protein biomarker further comprises prostate-specific antigen (PSA), endoglin, prostate-specific membrane antigen (PSMA), caveolin-1, interleukin-6, cluster of differentiation 147 (CD147), Transforming growth factor beta 1 (TGF-β1), human kallikrein-2, or combination thereof.

The methods described herein can be implemented on any system or instrument, including any manual, automated, or semi-automated system for immunoassays, flow cytometric analysis, or nucleic acid processing and sequencing. In some embodiments, the methods are single-plex or multi-plex. For example, a single sequencing method may detect more than one nucleic acid biomarker or a single flow cytometric method may detect more than one bioparticle surface marker.

Analyzing may include measuring or detecting at least one additional biomarker to those specifically disclosed. The additional biomarkers may include, for example, cancer biomarkers, prostate-specific antigen (PSA), circulating tumor cells (CTCs), circulating tumor DNA (ctDNA), and metabolites (e.g., metabolites of glycine synthesis and degradation, e.g., sarcosine).

In some embodiments, the at least one additional biomarker comprises at least one reference biomarker. The reference biomarker may comprise a ubiquitously expressed protein as a protein degradation control for the protein biomarkers. The at least one protein reference biomarker may account for total protein concentration, provide an indication of protein integrity, and/or confirm tissue of origin. In some embodiments, the reference protein biomarker comprises matriptase. In some embodiments, the reference protein biomarker comprises prostate-specific membrane antigen (PSMA). The reference biomarker may include a house-keeping gene to control for nucleic acid degradation in the sample. Reference nucleic acid biomarkers may confirm tissue of origin, for example, as based on methylation (e.g, prostate, sperm). Reference nucleic acid biomarkers may account for total DNA in a sample and/or total gene expression. Reference nucleic acid biomarkers include, but are not limited to, glyceraldehyde 3-phosphate dehydrogenase (GAPDH), beta-actin (ACTB), peptidylprolyl isomerase A (PPIA), peptidylprolyl isomerase B (PPIB), hypoxanthine phosphoribosyltransferase 1 (HPRT), microseminoprotein beta (MSMB), and developmental pluripotency antigen 5 (DPPA5). In some embodiments, the reference biomarker is glyceraldehyde 3-phosphate dehydrogenase (GAPDH).

c. Research, Screening, and Diagnosing of a Disease or Disorder

In some embodiments, systems and methods of the present disclosure include conducting research, screening, and/or diagnosing or assigning a risk level a disease or disorder (e.g., cancer) in a subject. The terms “diagnosing” and “diagnosis” as used herein refer to methods by which the skilled artisan can estimate and even determine whether or not a subject is suffering from a given disease or condition or may develop a given disease or condition in the future. In some embodiments, a diagnosis encompasses determining a prediction of progression of a disease or condition. For example, the diagnosis may provide a prediction of progression from early to late stage of the disease, progression from localized disease to wide-spread, or progression from non-aggressive to aggressive forms of cancer. The skilled artisan often makes a diagnosis on the basis of one or more diagnostic indicators, such as for example the biomarkers disclosed herein, which is indicative of the presence, severity, location, or absence of the condition.

In some embodiments, methods comprise providing a risk score based on evaluation of the biomarkers disclosed herein. The risk score may be indicative of whether a subject is likely to develop a given disease or disorder in the future. The risk score may be coordinated with other prognosis groupings for a particular disease or disorder (e.g., Gleason score for prostate cancer). The risk score may be coordinated with a prediction of progression of a disease or condition.

Providing a risk score may comprise applying an algorithm (e.g., a machine learning algorithm) to the biomarker results. The risk score may be weighted. For example, a weight may be assigned to any of the biomarkers based on their ability to predict risk or diagnose the disease or disorder in comparison to the other biomarkers by analyzed. Such weights may be determined by training a regression model (e.g., a linear regression model, a generalized linear model, a support vector regression model, a logistic regression model, a random forest regression model, a neural network model, regularized discriminant analysis, Naïve Bayes classification, etc.).

Thus, embodiments of the disclosure provide computer implemented methods for determining risk score using the results from the biomarkers. In some embodiments, the risk score is calculated with non-transient computer readable media or software. The software may be supplied in any electronic form such as a computer readable device, an internet download, or a web-based portal. The software may allow a proctor or user to view results in real-time, review results of previous samples, and view reports. The software may output data in the forms of images, graphs, charts, or raw values. The software may also be capable of calculating statistics and making comparisons between data sets.

The risk score may further be based on data other than the biomarkers disclosed herein. For example, the risk score may incorporate results from other biomarkers (e.g., cancer biomarkers, prostate-specific antigen (PSA), circulating tumor cells (CTCs), circulating tumor DNA (ctDNA), and other biomarkers), general health and wellness measurements, patient family history, and the like. As such, the methods may further comprise detecting at least one additional biomarker.

In some embodiments, the methods further comprise detecting at least one reference biomarker. The methods may further comprise detection of the expression levels of a ubiquitously expressed protein as a protein degradation control for the protein biomarkers. In some embodiments, the reference protein biomarker comprises matriptase. The methods may further comprise detection of the expression level or epigenetic modifications of a house-keeping gene to control for nucleic acid degradation in the sample. In some embodiments, the control nucleic acid biomarker is glyceraldehyde 3-phosphate dehydrogenase (GAPDH).

The methods described herein may be suitable for any disease or disorder in which biomarkers may provide an indication of risk or diagnosis.

In some embodiments, the methods described herein can be used to diagnose/detect a type of cancer. Types of cancers that can be detected/diagnosed using the methods of the present disclosure include, but are not limited to, lung cancer, melanoma, colon cancer, colorectal cancer, neuroblastoma, breast cancer, prostate cancer, renal cell cancer, transitional cell carcinoma, cholangiocarcinoma, brain cancer, non-small cell lung cancer, pancreatic cancer, liver cancer, gastric carcinoma, bladder cancer, esophageal cancer, mesothelioma, thyroid cancer, head and neck cancer, osteosarcoma, hepatocellular carcinoma, carcinoma of unknown primary, ovarian carcinoma, endometrial carcinoma, glioblastoma, Hodgkin lymphoma and non-Hodgkin lymphomas. In some embodiments, types of cancers or metastasizing forms of cancers that can be diagnosed or assessed by the methods of the present disclosure include, but are not limited to, carcinoma, sarcoma, lymphoma, germ cell tumor and blastoma. In some embodiments, the cancer is invasive and/or metastatic cancer (e.g., stage II cancer, stage III cancer or stage IV cancer). In some embodiments, the cancer is an early stage cancer (e.g., stage 0 cancer, stage I cancer), and/or is not invasive and/or metastatic cancer.

Assessment of a subject based on the biomarker profile or the determined risk score can be useful to separate subjects with good prognosis and/or low risk of developing a disease or disorder who will need no therapy or limited therapy from those more likely to develop a disease or disorder or suffer a recurrence of a disease or disorder who might benefit from more intensive treatments. As such, “making a diagnosis” or “diagnosing”, as used herein, is further inclusive of making a determination of a risk of developing a disease or disorder or determining a prognosis, which can provide for predicting a clinical outcome (with or without medical treatment), selecting an appropriate treatment (or whether treatment would be effective), or monitoring a current treatment and potentially changing the treatment, based on the identification and assessment of one or more biomarkers, as disclosed herein.

In some embodiments, the methods of the present disclosure include treating a subject having or suspected of having the disease or disorder or a subject at high risk of having the disease or disorder (e.g., a subject with cancer, with early-stage cancer or who is suspected of having cancer, or is at high risk of developing cancer). In some embodiments, the methods include determining the biomarker profile as provided herein and administering a treatment to a patient based on the results of determining the biomarker profile or the determined risk score. Suitable treatments can include administration of a pharmaceutical compound, a vaccine, performing a surgery, imaging the patient, and/or performing another test (e.g., a biopsy).

In some embodiments, the treatment comprises administration of an anti-cancer agent or chemotherapeutic. “Anti-cancer agent” or “chemotherapeutic,” as used herein, refers to any small molecule or other drug used in cancer treatment or prevention. Chemotherapeutics include, but are not limited to, cyclophosphamide, methotrexate, 5-fluorouracil, doxorubicin, docetaxel, daunorubicin, bleomycin, vinblastine, dacarbazine, cisplatin, paclitaxel, raloxifene hydrochloride, tamoxifen citrate, abemacicilib, afinitor, alpelisib, anastrozole, pamidronate, anastrozole, exemestane, capecitabine, epirubicin hydrochloride, eribulin mesylate, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, and tucatinib.

In some embodiments, the treating comprises active surveillance. As such, the methods described herein find use in classifying a patient as suitable for active surveillance During active surveillance the subject is monitored with additional screenings or tests for changes in overall health or changes directly related to disease progression. For example, in some embodiments, the methods comprise collecting or receiving a series of samples over a time period from the subject and detecting the one or more biomarkers in each of the series of samples and comparing any measurable change in the biomarkers over the period of time. In some embodiments, each of the series of samples may be used for diagnosing, detecting, or assigning a risk level for the subject at that given time point, as described in the methods herein, such that monitoring may comprise updating the diagnosis, risk level, and/or risk score, as described above, during the active surveillance period. Any changes over time may be used to predict risk of developing the disease or disorder or to determine whether to initiate or continue any treatment or active surveillance.

In some embodiments, the methods of the present disclosure can be used as part of clinical screening, a method of prognosis assessment, a method of monitoring the results of therapy, a method of imaging a patient or subject, and a method for drug screening and development. Any changes in the one or more biomarkers over the time period following treatment can be used to predict clinical outcome, determine whether to initiate or continue the treatment of the disease, and determine whether the initial treatment therapy is effectively treating the cancer. Thus, in some embodiments, the subject may begin a treatment and, following an additional round of detecting the one or more biomarkers, the effectiveness of the treatment may be assessed, a determination of continuation of the original treatment or cessation of the treatment made be made, or a second treatment regimen in addition or in place of the original treatment may be started. Additionally, samples may be taken and analyzed for the one or more biomarkers during the course of a single treatment regimen to monitor the duration of treatment which facilitates effective treatment.

For example, a first time point can be selected prior to initiation of a treatment and a second time point can be selected at some time after initiation of the treatment. Detection of the one or more biomarkers in each of the samples taken from different time points and qualitative and/or quantitative differences can be noted. A change in the one or more biomarkers from the different samples can be correlated with prognosis, determining treatment efficacy, and/or progression of the disease or disorder in the subject.

In some embodiments, the methods and compositions of the invention are for treatment or diagnosis of disease at an early stage, for example, before symptoms of the disease appear. Thus, the disclosed methods may be used to monitor delay of symptomatic disease. In some embodiments, the methods and compositions of the invention are for treatment or diagnosis of disease at a clinical stage. Thus, the disclosed methods may be used to monitor duration and frequency of decreases in symptoms of the disease or disorder.

Capturing details of the biological activity through minimally-invasive techniques can also inform precision medicine or personal medicine. Precision medicine is an approach for disease treatment and prevention that takes into account individual variability in genes, environment, and lifestyle for each person. For example, the disclosed methods may be particular useful when serial biopsies may be too difficult to achieve or where drug efficacy for a subject is being tested over time. The disclosed methods facilitate merging of information that is specific to the subject or the subject's disease or disorder (e.g., genetic information, biomarker (e.g., protein and nucleic acid) information, general health information (e.g., age, weight, sex, medical history, family medical history)) with treatment options that are compatible with the subject's individual genetic profile. As such, in some embodiments, the methods may further comprise selecting or adapting a treatment regime of an individual subject based on the detected biomarkers specific to the disease or disorder and other genetic, environmental and lifestyle characteristics of the subject.

### 3) Kits

In various embodiments, the present invention provides a kit for identifying one or more biomarkers in a sample. The kit may comprise one or more internal standards suitable for any one or more of immunoassays, amplification reactions, flow cytometry or any other analysis methods and reagents, devices, and instructions for sample processing, preparation, and analysis.

In some embodiments, the kit comprises one or more reagents for detection of a nucleic acid biomarker, including but not limited to sequencing reagents (e.g., primers, probes, nucleotides, buffers, control nucleic acid sequences, polymerases, etc.), restriction endonucleases, enzymes for oxidizing methylated nucleotides and reducing the oxidized methylated nucleotide and the like.

In some embodiments, the kit comprises one or more reagents for detection of a protein biomarker, including but not limited to antibodies, blocking reagents and buffers, detection reagents (e.g, detectable labels, enzymes, and dyes) and the like.

Individual member components of the kits may be physically packaged together or separately. For example, the base and the reservoir may be packaged together as a single device and the cap may be packaged together with the device or separately.

The kits can also comprise instructions for using the components of the kit. The instructions are relevant materials or methodologies pertaining to the kit. The materials may include any combination of the following: background information, list of components, brief or detailed methods for using the system, trouble-shooting, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods.

### 4) EXAMPLES

### Example 1

Semen samples were collected in a multi-center biorepository clinical trial of men aged 50 or older. Fourteen sites participated across the U.S. A single sample was collected from each study participant and assigned to four cohorts as defined in Table 1. Semen samples were processed and AMACR protein level was quantified using an ELISA assay.

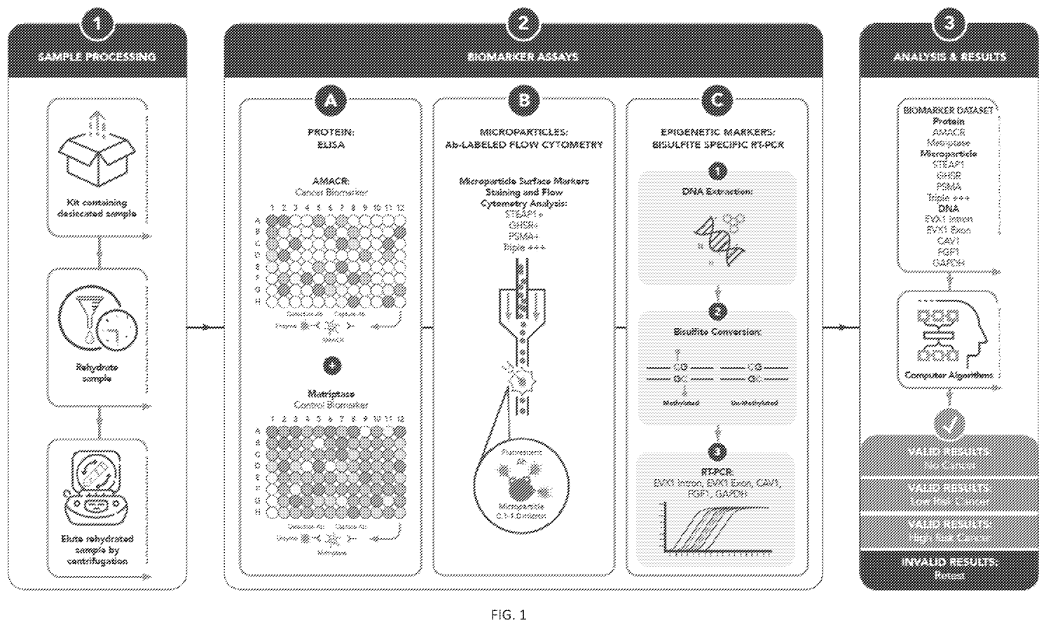

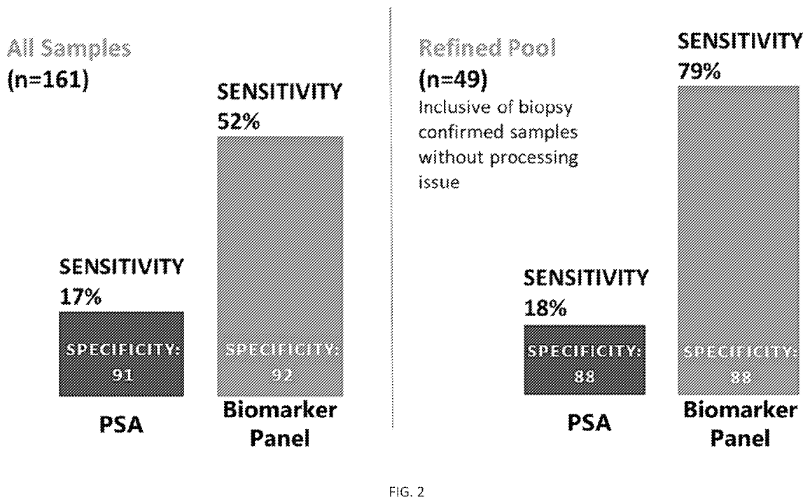

Bioparticle quantity and surface marker profiles (PSMA, STEAP1 and GHSR1a) were characterized using antibody-stained flow cytometry. DNA methylation profiles of CAV1, EVX1, and FGF1 were characterized by methyl-specific PCR assay (FIG. 1). Data normalization, univariate and multivariate LDA analysis was conducted to determine sensitivity and specificity of prostate cancer diagnosis between PSA alone and the test panel. A subset of samples was analyzed to include only those without processing issues (45% of samples) and those with a biopsy confirmed ‘Cancer’ or ‘No Cancer’ status (excluding cohort 1), shown in FIG. 2.

TABLE 1

| Cohort | N | PSA (ng/mL) | Biopsy Result | Gleason Score | Risk |
|---|---|---|---|---|---|
| 1 | 64 | <2.0 | NA | NA | Normal Sample |
| 2 | 51 | ≥2.0 | − | NA | BPH Elevated PSA |
| 3 | 40 | ≥2.0 | + | 6 to 7 (3 + 4) | Low |
| 4 | 6 | ≥2.0 | + | 7 (4 + 3) to 10 | High |

The disclosed biomarker panel encompassing DNA methylation, protein, and bioparticle markers greatly improved prostate cancer detection sensitivity over PSA alone or any single marker alone, 18% compared to 79%. However, the improved sensitivity was manifested strongest in samples without any processing issues and those with biopsy confirmation. Overall, there is a four-fold increase in sensitivity of cancer detection when compared to diagnosis made using PSA alone.

### Example 2

Two hundred thirty-nine patient semen samples were tested using a desiccation processing methods and a panel of 11 biomarkers to demonstrate the sensitivity and specificity, for prostate cancer detection and prostate cancer differentiation.

Stages of prostate cancer progression are assessed through histology of biopsy tissues and graded with a Gleason Score. Indolent cancer has a Gleason Score of 7 (3+4) and below while aggressive cancer has a Gleason Score 7 (4+3) and above. Four cohorts of men aged 50-70 participated under the criteria that the participants had not had a prostate biopsy 30 days prior to seminal fluid collection, had not undergone prostate cancer treatments, and had not had a vasectomy.

Samples for further analysis were classified into the following cohorts.
1. Cohort 2—Men with potential elevated PSA values and negative prostate biopsy results within the previous 12 months indicating ‘no prostate cancer.’
2. Cohort 3—Men with positive biopsy results indicating ‘low risk prostate cancer’ (Gleason scores of 6 and 7 (3+4))
3. Cohort 4—Men with positive biopsy results indicating ‘high risk prostate cancer’ (Gleason scores of 7 (4+3) and higher).
   Alternative classifications are also possible based on Gleason Scores and ‘intermediate risk prostate cancer.’

Subjects enrolled into the study as Cohort 1 (low, normal prostate specific antigen (PSA) values) provided a seminal fluid sample to their enrolling site. Additional subjects who were referred to prostate biopsy (Cohorts 2-4) were consented and enrolled into the study and provided a seminal fluid sample to their recruiting site before their biopsy. If the subject was unable to provide the sample ahead of biopsy, samples were subsequently collected at least 30 days post-biopsy. The results of the biopsy were combined with other prostate history and demographic/medical history (e.g., age, race/ethnicity, family history, medical history, medications, past PSA results, digital rectal exam (DRE) results and notes, prostate biopsy results, prostate cancer diagnosis and staging results, family history of prostate cancer, and vasectomy status). Upon receiving results of the prostate biopsy, the patient was placed into their respective cohort (Cohorts 2, 3, or 4) based on the biopsy results.

A research-specific single semen sample was collected by the patient in each cohort on site or at home and brought back to the clinical site for processing and storage. Each sample was transferred from collection cup into a 15 mL conical vial and frozen for shipment to the testing site. Samples were frozen, generally at −80° C., at the testing site until use.

Desiccation and Rehydration Sample Processing Each sample for analysis was thawed at room temperature and processed through a desiccation and rehydration method. Whatman/Cytiva CF7 filter paper of 3 cm×0.5 cm dimension were placed inside the tube containing thawed seminal fluid sample for absorption into the filter paper for 10 minutes. After sample was fully absorbed, it was transferred using single use forceps to a designed desiccation chamber consisting of an airtight 100 ml collection cup with 10 g of silica bead desiccant and a 3D printed plastic filter paper holder allowing filter paper to dry within 10 hours. Samples were desiccated overnight up to 24 hours and then rehydrated the following day. The filter paper was transferred to a new 2 ml tube and 1.8 ml of PBS was added to the tube, vortexed for 5 seconds and incubated at 65° C. for 30 minutes. The filter paper was then transferred to a 15 ml tube containing an insert filter of 100 um mesh and centrifuged at 3500 rpm for 2 minutes to elute rehydrated sample. The sample was then transferred to a new tube and aliquoted to different volumes based on the assays performed. All aliquots were frozen until analysis.

Preliminary analysis showed that desiccation recovered the target biomarkers as well as or better than traditional methods, e g., centrifugation, particularly in samples that are viscous, low volume, and have possible red blood cells. No difference in preservation of the biomarkers was seen when samples were desiccated and stored at ambient temperature for up to 7 days compared to those methods in which the samples were stored frozen (−20° C.)

A panel of 11 biomarkers were analyzed for each subject sample.

### Protein Detection Through ELISA

AMACR: The semen samples were diluted 10-fold and processed using an AMACR ELISA Kit (LSBio LS-F21243) based on manufacturer's instructions.

Matriptase: The semen samples were diluted 2-fold and processed using a Matriptase ELISA Kit (LSBio/LS-49987) based on manufacturer's instructions.

### Bioparticle (MP) Detection Through Flow Cytometry

The semen samples were diluted 2-fold and fluorescently-labeled antibodies corresponding to each of the surface markers were added to the diluted sample at 0.0125 mg/mL.

### Methylation Profiling of Cancer Associated Genes Through qRT-PCR

Circulating cell free DNA from semen samples were processed using the Maxwell® RSC ccfDNA Plasma Kit and the Maxwell® RSC Instrument from Promega Corporation following manufacturer's instructions. The resulting cell free DNA was bisulfite converted (EpiTect Fast 96 Bisulfite Conversion Kits) and amplified with methylation specific primers and probes to determine the methylation status of the original sequence.

Statistical Analysis All outcomes are presented using descriptive statistics, e.g., mean and standard deviation (SD), skewed distributions by the median and interquartile range (IQR). Binary and categorical variables are presented using counts, percentages, selectivity & specificity, and contingency table. MATLAB statistical methods were used for conventional statistical analysis and nominal regression analysis. A precision-based method evaluated the power of ‘precision of effect’ estimates. Descriptive statistical analysis was applied to the data, including the numbers and percentages for categorical variables. The number, means, standard deviation, medians, minimums, and maximums for continuous variables are provided. Exploratory analysis may also be performed, supplemented by calculation.

Of the two hundred thirty-nine patient semen samples, a total of two hundred nineteen were processed through the entire biomarker panel. The excluded samples included those without accurate cohort information, those which encountered processing and handling errors, those without enough reference protein (matriptase) or DNA (GAPDH), and those with contaminated samples (e.g., blood in sample).

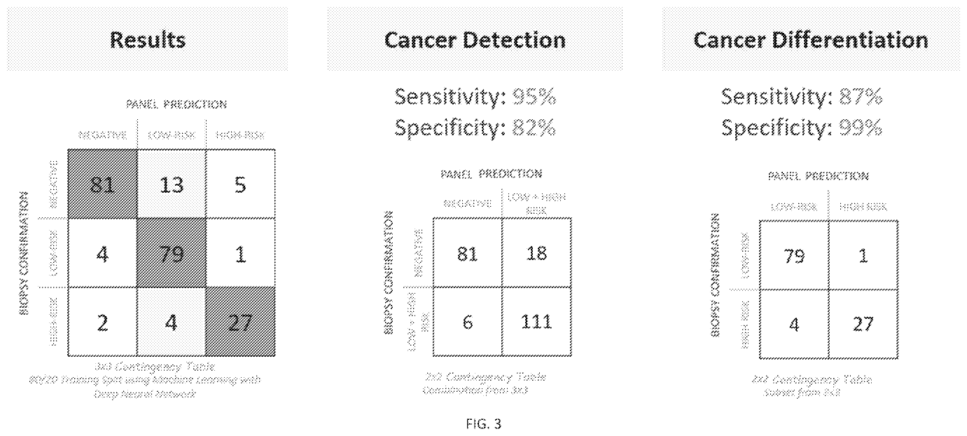

As shown in FIG. 3, the data shows a high ability to differentiate low-risk versus high-risk cancer. This data was processed using a Deep Neural Network (DNN) model with an 80/20 data split. Mathematically, one can apply the power law (or Pareto distribution) to approximate the 80/20 rule for a particular dataset. It has been also shown that many natural phenomena have been shown to exhibit such a distribution. Hence, it was assumed that 80% of the data samples should be able to describe the functional relation between biomarkers and be able to predict the results for the remaining 20%.

In order to compute the sensitivity (True Positive/(True Positive+False Negative)) and specificity (True Negative/(True Negative+False Positive)) for cancer detection, a 2×2 contingency table modified from the 3×3 contingency table was built (FIG. 3, middle). For this reason, true ‘low-risk’ and ‘high-risk’ predictions were summed including false ‘high-risk’ and ‘low-risk’ into the ‘low+high risk’ predictions (leading to 111 cases overall). The off-diagonal blocks were also summed leading to 18 (False positive ‘Low+High Risk’ cases) and 6 (False Negative) cases. Using the above explained expressions, the sensitivity and specificity were computed using a modified 2×2 contingency table leading to 95% (100*111/117) and 82% (100*81/99) respectively. The same can be done to compute sensitivity and specificity for the cancer of low-risk and high-risk cancer patients, leading to 87% (100*27/31) and 99% (100*79/80), respectively (FIG. 3, right). In this case, of 80 cases of low-risk cancer only 1 was falsely predicted as high-risk, and of 31 high-risk cases, 4 were incorrectly classified as low-risk. This example is representative of patients on active surveillance, where model could be used to predict who could be safely kept on active surveillance, and who should receive treatment. With 79 of 80 low-risk patients predicted to have low-risk disease using the model, the negative-predictive value (NPV) is 98.8% and the positive predictive value (PPV) is calculated to be 87.1%.

As a result of the biomarker panel in comparison to PSA tests, 94 biopsies would have been avoided entirely and 83 biopsies would have been replaced with active surveillance. In total of the 219 patients, 82% of the biopsies (177 total) would not have been completed.

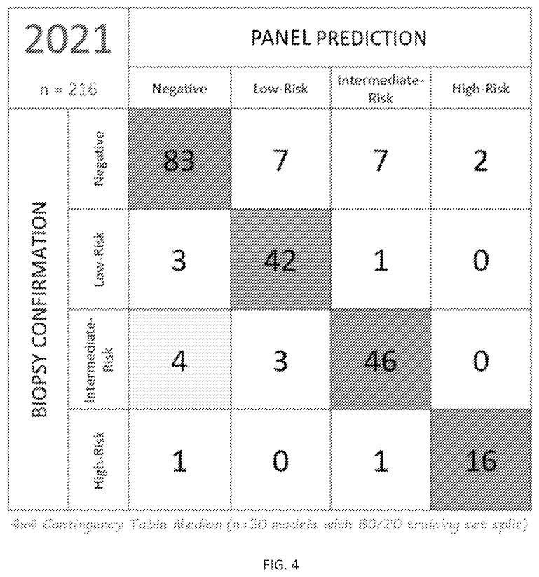

Using a similar analysis, the sample were classified by low risk, intermediate risk, and high risk (FIG. 4). The sensitivity and specificity were computed for each of the classifications (Table 2).

TABLE 2

| | Sensitivity | Specificity |
|---|---|---|
| Negative | 83.3% | 92.9% |
| Low Risk | 91.3% | 93.5% |
| Intermediate Risk | 86.7% | 94.0% |
| High Risk | 88.8% | 98.8% |
| Average | 87.7% | 94.8% |

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A method comprising:
collecting or receiving a semen sample from a subject;
detecting one or more biomarkers in the semen sample; and
diagnosing, detecting, or assigning a risk level for a disease or disorder in the subject.

2. The method of claim 1, wherein the one or more biomarkers comprise at least one protein biomarker, at least one nucleic acid biomarker, or a combination thereof.

3. The method of claim 2, wherein the at least one nucleic acid biomarker is from cellular DNA, cell free DNA, circulating tumor cell DNA, RNA, or a combination thereof.

4. The method of claim 2 or 3, wherein the at least one nucleic acid biomarker is selected from the group consisting of: EVX1, the gene for homeobox even-skipped homolog protein 1, FGF1,the gene for fibroblast growth factor 1, CAV1, the gene for caveolin-1, or a combination thereof.

5. The method of any of claims 2-4, wherein the at least one nucleic acid biomarker comprises an epigenetic marker.

6. The method of claim 5, wherein detecting the at least one nucleic acid marker comprises determination of differential methylation of the at least one nucleic acid biomarker.

7. The method of claim 6, wherein differential methylation is determined by bisulfite specific nucleic acid amplification, enzymatic conversion, immunocapture, or a combination thereof.

8. The method of claim 6 or 7, wherein detecting the at least one nucleic acid marker comprises determining differential methylation of EVX1, CAV1, FGF1, or a combination thereof.

9. The method of any of claims 6-8, wherein detecting the at least one nucleic acid marker comprises determining differential methylation of each of EVX1, CAV1, and FGF1.

10. The method of any of claims 2-9, wherein the at least one protein biomarker is selected from the group consisting of: alpha-methylacyl-CoA racemase (AMACR), Six Transmembrane Epithelial Antigen Of The Prostate 1 (STEAP1), Growth Hormone Secretagogue Receptor (GHSR), prostate-specific membrane antigen (PSMA), and combinations thereof.

11. The method of any of claims 2-10, wherein detecting the at least one protein biomarker comprises determining the expression level of the at least one protein biomarker in an immunoassay.

12. The method of any of claims 2-11, wherein the at least one protein biomarker is a bioparticle surface protein.

13. The method of claim 12, further comprising quantifying bioparticles in the sample.

14. The method of claim 12 or 13, wherein detecting the at least one protein biomarker comprises quantifying bioparticles with STEAP1, GHSR, PSMA, or a combination thereof.

15. The method of any of claims 12-14, wherein detecting the at least one protein biomarker comprises quantifying bioparticles with STEAP1, GHSR, and PSMA.

16. The method of any of claims 1-15, wherein detecting one or more biomarkers in the semen sample comprises detecting the expression level of AMACR, quantifying bioparticles with STEAP1, GHSR, and PSMA, determining differential methylation of each of EVX1, CAV1, and FGF1, and any combination thereof.

17. The method of any of claims 1-16, wherein detecting one or more biomarkers in the semen sample comprises detecting the expression level of AMACR, quantifying bioparticles with STEAP1, GHSR, and PSMA, and determining differential methylation of each of EVX1, CAV1, and FGF1.

18. The method of any of claims 1-17, further comprising detecting one or more control biomarkers.

19. The method of any of claims 1-18, further comprising adding a buffer or buffer system to the semen sample.

20. The method of any of claims 1-18, further comprising drying the semen sample.

21. The method of claim 20, wherein the semen sample is dried on a capture material.

22. The method of claim 21, wherein the capture material comprises filter paper, cotton linter, or a combination thereof.

23. The method of any of claims 20-22, wherein the drying is carried out by a device comprising a capture material.

24. The method of claim 23, wherein the device comprises one or more sample regions each comprising capture material.

25. The method of claim 24, wherein the capture material in each of the one or more sample regions is the same or different.

26. The method of any of claims 23-25, wherein the drying and collecting is carried out using by a single device.

27. The method of any of claims 23-26, wherein the device further comprises a desiccant in vaporous communication with the semen sample and/or the capture material.

28. The method of any of claims 20-27, further comprising rehydrating dried semen sample.

29. The method of claim 28, wherein rehydrating comprises:
incubating the capture material with buffer or solvent at about 4° C. to about 90° C.; and separating capture material from rehydrated sample.

30. The method of any of claims 1-29, further comprising shipping the semen sample.

31. The method of claim 30, wherein shipping the semen sample is carried out using the same device as the drying and collecting.

32. The method of claim 30 or 31, wherein the semen sample at least partially dries during shipping.

33. The method of any of claims 1-32, wherein diagnosing or assigning a risk level for a disease or disorder comprises providing a risk score based on evaluation of the one or more biomarkers.

34. The method of any of claims 1-33, wherein the disease or disorder comprises cancer.

35. The method of any of claims 1-34, wherein the disease or disorder comprises prostate cancer or testicular cancer.

36. The method of any of claims 1-35, further comprising treating a subject.

37. A method comprising:
collecting or receiving a sample from a subject;
detecting one or more biomarkers in the sample,
wherein the one or more biomarkers comprise:
at least one protein biomarker comprising alpha-methylacyl-CoA racemase (AMACR),
at least one bioparticle surface marker selected from the group consisting of STEAP1, GHSR, PSMA, and combinations thereof,
at least one nucleic acid biomarker selected from the group consisting of EVX1, CAV1, FGF1, or a combination thereof, or
any combination thereof.

38. The method of claim 37, wherein the at least one nucleic acid biomarker is from cellular DNA, cell free DNA, circulating tumor cell DNA, RNA, or a combination thereof.

39. The method of claim 37 or 38, wherein the at least one nucleic acid biomarker is selected from the group consisting of: EVX1, CAV1, FGF1, or a combination thereof.

40. The method of any of claims 37-39, wherein the at least one nucleic acid biomarker comprises an epigenetic marker.

41. The method of any of claims 37-40, wherein detecting the at least one nucleic acid biomarker comprises determining differential methylation of the at least one nucleic acid biomarker.

42. The method of claim 41, wherein differential methylation is determined by bisulfite specific nucleic acid amplification, enzymatic conversion, immunocapture, or a combination thereof.

43. The method of any of claims 37-42, wherein detecting the at least one nucleic acid biomarker comprises determining differential methylation of EVX1, CAV1, FGF1, or a combination thereof.

44. The method of any of claims 37-43, wherein detecting the at least one nucleic acid biomarker comprises determining differential methylation of each of EVX1, CAV1, and FGF1.

45. The method of any of claims 37-44, wherein the at least one protein biomarker comprises alpha-methylacyl-CoA racemase (AMACR).

46. The method of any of claims 37-45, wherein detecting the at least one protein biomarker comprises determining the expression level of the at least one protein biomarker in an immunoassay.

47. The method of any of claims 37-46, wherein the at least one bioparticle surface marker comprises STEAP1, GHSR, PSMA, and combinations thereof.

48. The method of any of claims 37-47, further comprising quantifying bioparticles in the sample.

49. The method of any of claims 37-48, wherein detecting the at least one bioparticle surface marker comprises quantifying bioparticles with STEAP1, GHSR, PSMA, or a combination thereof.

50. The method of any of claims 37-49, wherein detecting the at least one bioparticle surface marker comprises quantifying bioparticles with STEAP1, GHSR, and PSMA.

51. The method of any of claims 37-50, wherein detecting one or more biomarkers in the sample comprises determining the expression level of AMACR, quantifying bioparticles with STEAP1, GHSR, and PSMA, determining differential methylation of each of EVX1, CAV1, and FGF1, and any combination thereof.

52. The method of any of claims 37-51, detecting one or more biomarkers in the sample comprises determining the expression level of AMACR, quantifying bioparticles with STEAP1, GHSR, and PSMA, and determining differential methylation of each of EVX1, CAV1, and FGF1.

53. The method of any of claims 37-52, further comprising adding a buffer or buffer system to the sample.

54. The method of any of claims 37-53, further comprising drying the sample.

55. The method of claim 54, wherein the sample is dried on a capture material.

56. The method of claim 55, wherein the capture material comprises wherein the capture material comprises filter paper, cotton linter, or a combination thereof.

57. The method of claim 55 or 56, wherein the drying is carried out by a device comprising a capture material.

58. The method of claim 57, wherein the device comprises a one or more sample regions each comprising capture material.

59. The method of claim 58, wherein the capture material in each of the one or more sample regions is the same or different.

60. The method of any of claims 57-59, wherein the drying and collecting is carried out using by a single device.

61. The method of claim 60, wherein the device further comprises a desiccant in vaporous communication with the sample and/or the capture material.

62. The method of any of claims 54-61, further comprising rehydrating dried sample

63. The method of claim 62, wherein rehydrating comprises:
incubating the capture material with buffer at about 4° C. to about 90° C.; and
separating capture material from rehydrated sample.

64. The method of any of claims 37-63, further shipping the sample.

65. The method of claim 64, wherein shipping the sample is carried out using the same device as the drying and collecting.

66. The method of claim 65, wherein the sample at least partially dries during shipping.

67. The method of any of claims 37-66, further comprising diagnosing, detecting, or assigning a risk level for a disease or disorder in the subject.

68. The method of claim 67, wherein diagnosing or assigning a risk level for a disease or disorder comprises providing a risk score based on evaluation of the one or more biomarkers.

69. The method of claim 67 or 68, wherein the disease or disorder comprises cancer.

70. The method of any of claims 67-69, wherein the disease or disorder comprises prostate cancer.

71. The method of any of claims 67-70, further comprising treating the subject.

72. The method of any of claims 37-71, wherein the sample is viscous.

73. The method of any of claims 37-72, wherein the sample is non-homogeneous.

74. The method of any of claims 37-73, wherein the sample comprises semen, synovial fluid, mucus, pus, secretions, or combinations thereof.

75. The method of any of claims 37-74, wherein the sample is a semen sample.

76. A method for processing or preparing a biological sample for analysis comprising:
collecting or receiving a biological sample from a subject;
drying the biological sample; and
rehydrating dried biological sample,
wherein the sample is viscous, non-homogeneous or a combination thereof.

77. The method of claim 76, wherein the biological sample is dried on a capture material.

78. The method of claim 77, wherein the capture material comprises wherein the capture material comprises filter paper, cotton linter, or a combination thereof.

79. The method of claim 77 or 78, wherein the drying is carried out by a device comprising a capture material.

80. The method of claim 79, wherein the device comprises one or more sample regions each comprising capture material.

81. The method of claim 80, wherein the capture material in each of the one or more sample regions is the same or different.

82. The method of any of claims 79-81, wherein the drying and collecting is carried out using by a single device.

83. The method of any of claims 79-82, wherein the device further comprises a desiccant in vaporous communication with the biological sample and/or the capture material.

84. The method of any of claims 76-83, wherein rehydrating comprises:
incubating the capture material with buffer at about 4° C. to about 90° C.; and
separating capture material from rehydrated biological sample.

85. The method of any of claims 76-84, further comprising shipping the biological sample.

86. The method of claim 85, wherein shipping the sample is carried out using the same device as the drying and collecting.

87. The method of claim 85 or 86, wherein biological sample at least partially dries during shipping.

88. The method of any of claims 76-87, further comprising analyzing rehydrated biological sample.

89. The method of claim 88, wherein the analyzing comprises detecting one or more biomarkers in the rehydrated biological sample.

90. The method of claim 89, wherein the one or more biomarkers comprise at least one protein biomarker, at least one nucleic acid biomarker, or a combination thereof.

91. The method of claim 90, wherein the at least one nucleic acid biomarker is from cell free DNA, circulating tumor DNA, RNA, or a combination thereof.

92. The method of claim 90 or 91, wherein the at least one nucleic acid biomarker is selected from the group consisting of: EVX1, CAV1, FGF1, or a combination thereof.

93. The method of any of claims 90-92, wherein the at least one nucleic acid biomarker comprises an epigenetic marker.

94. The method of claim 93, wherein detecting the at least one nucleic acid marker comprises determination of differential methylation of the at least one nucleic acid biomarker.

95. The method of claim 94, wherein differential methylation is determined by a bisulfite specific nucleic acid amplification method.

96. The method of claim 94 or 95, wherein detecting the at least one nucleic acid marker comprises determining differential methylation of EVX1, CAV1, FGF1, or a combination thereof.

97. The method of any of claims 94-96, wherein detecting the at least one nucleic acid marker comprises determining differential methylation of each of EVX1, CAV1, and FGF1.

98. The method of any of claims 90-97, wherein the at least one protein biomarker is selected from the group consisting of: alpha-methylacyl-CoA racemase (AMACR), STEAP1, GHSR, PSMA, and combinations thereof.

99. The method of any of claims 90-98, wherein detecting the at least one protein biomarker comprises determining the expression level of the at least one protein biomarker in an immunoassay.

100. The method of any of claims 90-99, wherein the at least one protein biomarker is a bioparticle surface protein.

101. The method of claim 100, further comprising quantifying bioparticles in the sample.

102. The method of claim 100 or 101, wherein detecting the at least one protein biomarker comprises quantifying bioparticles with STEAP1, GHSR, PSMA, or a combination thereof.

103. The method of any of claims 100-102, wherein detecting the at least one protein biomarker comprises quantifying bioparticles with STEAP1, GHSR, and PSMA.

104. The method of any of claims 89-103, wherein detecting one or more biomarkers in the rehydrated biological sample comprises detecting the expression level of AMACR, quantifying bioparticles with STEAP1, GHSR, and PSMA, determining differential methylation of each of EVX1, CAV1, and FGF1, and any combination thereof.

105. The method of any of claims 89-104, wherein detecting one or more biomarkers in the rehydrated biological sample comprises detecting the expression level of AMACR, quantifying bioparticles with STEAP1, GHSR, and PSMA, and determining differential methylation of each of EVX1, CAV1, and FGF1.

106. The method of any of claims 89-105, further comprising detecting one or more control biomarkers.

107. The method of any of claims 89-106, further comprising diagnosing or assigning a risk level for a disease or disorder in the subject.

108. The method of claim 107, wherein diagnosing, detecting, or assigning a risk level for a disease or disorder comprises providing a risk score based on evaluation of the one or more biomarkers.

109. The method of any of claims 76-108, wherein the biological sample comprises semen, synovial fluid, mucus, pus, secretions, or combinations thereof.

110. The method of any of claims 76-109, wherein the biological sample is a semen sample.
